(19) United States
(12) Patent Application Publication
(10) Pub. No.: US 2024/0307233 A1
(43) Pub. Date: Sep. 19, 2024

(54) ELASTIC ADHESIVE COMPRESSION DRESSING

(71) Applicant: RETIYA MEDICAL SOLUTIONS, DEERFIELD BEACH, FL (US)

(72) Inventors: Sody Naimer, Neve Dkalim (IL); David Rand, Deerfield Beach, FL (US); Tuvia Lewkowicz, Jerusalem (IL)

(21) Appl. No.: 18/394,634

(22) Filed: Dec. 22, 2023

Related U.S. Application Data
(60) Provisional application No. 63/435,080, filed on Dec. 23, 2022.

(51) Int. Cl. A61F 13/02; A61F 13/00

(52) U.S. Cl. CPC A61F 13/0273; A61F 2013/00106; A61F 2013/00127; A61F 2013/00468

(57) ABSTRACT

A compression dressing includes an elastic bandage strip that is elongated, transparent and self-adhering, and has a first outer side and a second inner side, with a first unextended state and a second elastically extended state. A self-adhesive surface is provided on the first outer side of the elastic bandage strip. An absorbent pad at a terminal end of the elastic bandage strip is applied to a wound, and the remainder of the elastic bandage strip may be wrapped around the absorbent pad and a body part to hold the compression dressing in place and to exert a compressive force on the wound. An elastic bandage strip and/or the absorbent pad includes one or more of spaced apart blood migration markings, compression markings, and a skin adhesive. Methods for applying a compression dressing to a wound to both observe the status and treat the wound are also disclosed.

### CROSS-REFERENCE TO RELATED APPLICATIONS

This application claims priority to U.S. 63/435,080 filed on Dec. 23, 2022, entitled “Elastic Adhesive Compression Dressing”, the entire disclosure of which incorporated herein by reference.

### FIELD OF THE INVENTION

The present invention relates to a wound dressing effective in rapidly controlling bleeding from a wound and, more particularly, to a wound dressing constructed and packaged such that the wound may be dressed using one hand.

### BACKGROUND OF THE INVENTION

Bleeding control is a major consideration in the emergency treatment of acute trauma. Rapid and effective application of compression, at the scene of the traumatic incident until arrival at a medical facility, leads to decreased loss of blood from wounds and improves the chances of survival of the victim(s). Simultaneous therapy and observation of bleeding status can provide improved outcomes.

Traditional methods of bleeding control include direct compression, applied over the wound surface, usually employing an absorbent layer, and the tourniquet, tightly applied proximally to the wound. Direct compression is often effective in controlling bleeding, achieving high local pressures. Direct compression, however, is impractical in many emergency situations due to the need for continuous application by emergency care providers, thus neutralizing the care provider, denying performance of any other tasks. Direct compression cannot sustain the required constant local pressure, resulting in an increased tendency of the absorbent dressings to become saturated with blood. In addition, heavy direct compression may aggravate damage to other wounded tissues, such as fractures, and is inconvenient to apply over irregularly shaped or sensitive body parts.

The tourniquet, consisting of an elastic material bound tightly around the wounded body part, proximally to the wound, is applied with sufficient constricting force to cause ischemia distally to the site of application. This highly effective and widely used method of bleeding control may be left unattended, however, the induced ischemia is both extremely painful to the victim and commonly the cause of soft tissue and neurological damage to the distal body parts. Prevention of these undesired effects of tourniquet application is achieved by relieving the pressure at designated intervals, compromising the effectiveness and requiring additional attention by emergency care providers.

Elastic compression bandages, applied directly over the wound surface, are capable of providing significant compression and may be used in wound dressings. Elastic bandages are typically constructed of an elastomeric material, or a composite of elastic and non-elastic materials, which may be extended to provide constriction of the underlying surface. However, these fabric wraps, being non-adhesive, tend to shift with motion, compromising their comfort and compression capabilities, and therefore need to be fastened or bonded to an adhesive. Such wraps also lack absorbent qualities and lack transparency.

U.S. Pat. Nos. 3,575,782 and 4,984,584 to Hansen, and U.S. Pat. No. 4,699,133 to Schafer describe elastomeric, self-adhering compression bandages capable of achieving effective compression forces when applied over a dressed wound, but lacking absorbent properties. Many other different bandages have been proposed. Absorbent elastic wraps have been employed in compression bandages. U.S. Pat. No. 4,707,398 to Boggs, U.S. Pat. No. 4,720,415 to Vander Weilen, U.S. Pat. No. 5,230,701 to Meyer, and U.S. Pat. No. 4,957,795 to Reidel employ absorbent, elastic bandages which afford both compression and exudate retention. However, the materials employed are non-self-adherent, requiring fastening and are prone to slippage when saturated with fluid.

Other configurations are disclosed in U.S. Pat. Nos. 2,858,830; 3,490,448; 3,888,247; 3,927,665; 3,625,209; 3,421,502; 4,005,709; and 4,377,159, and in BE 00522994 and GB00487743. U.S. Pat. No. 5,939,339 to Delmore et al. describes a porous, elastic, self-adherent bandage flexibly attached to an absorbent layer. The self-adhering elastic is produced from elastic or spandex yarns bonded between porous fibrous webs or fabric, or a web of elastomeric polyurethane fibers. The absorbent layer covers all but a small portion of the elastic bandage's width along a major portion of the bandage's length. Thus, when applied, most of the absorbent pad is not in direct contact with the wound surface, and little self-adhesive bandage surface remains exposed, disposing the bandage to slippage and poor compression with movement and continued bleeding.

Naimer U.S. Pat. No. 6,573,419 discloses an elastic adhesive wound dressing that includes a self-adhering elastic bandage strip for exerting a compressive force when wrapped around a body part that is sufficient to hold the compression dressing in place, and an absorbent pad affixed to an inner side of a terminal portion or to a terminal end of the self-adhering bandage strip. The disclosure of this patent is hereby incorporated fully by reference.

There is a continuing need for a wound dressing effective in bleeding control and that is readily self-applied, particularly in emergency situations where the patient might be without assistance, such as battlefields or remote locations. There is also a need for a wound dressing that is readily and properly applied by non-medical personnel or relatively unskilled persons. There is a further need to provide treatment for bleeding while observing the status of the bleeding. There is a need as well for a device that accomplishes prevention of exsanguination while not causing complete distal ischemia, reducing pain and risk of irreversible tissue damage possibly requiring amputation.

### SUMMARY OF THE INVENTION

A compression dressing includes an elastic bandage strip that is elongated, transparent and self-adhering, and has a first outer side and second inner side. The elastic bandage strip has a first unextended state and a second elastically extended state. A self-adhesive surface is provided on the elastic bandage strip. The self-adhesive surface can be a property of the material making up the elastic bandage strip or an adhesive coating applied to either the first outer side or second inner side that interacts with the opposing adjacent side after wrapping. The self-adhesive surface is capable of adhering the first outer side of the elastic bandage strip to the second inner side of an adjacent portion of the elastic bandage strip when wrapped.

An absorbent pad of lesser length than the elastic bandage strip is affixed to a terminal end of the elastic bandage strip. The elastic bandage strip is of sufficient length such that the absorbent pad may be applied to a wound on a body part, and the remainder of the elastic bandage strip may be wrapped around the adsorbent pad and the body part to hold the compression dressing in place and to exert a compressive force on the wound for a period of time to permit a therapeutic effect to the wound. At least one of the elastic bandage strip and the adsorbent pad includes spaced apart blood migration markings for indicating the migration of blood across the absorbent pad, the blood migration markings being visible through wrappings of the elastic bandage strip. The blood migration markings can include a grid of columns and rows. The compression dressing can further include indicia for identifying individual ones of the columns and rows.

The elastic bandage strip can include a first terminal end affixed to the absorbent pad, and a second terminal end. The compression dressing can further include a fastening device secured nearer to the first terminal end than to the second terminal end, for engaging the second terminal end to the first terminal end and preventing unwrapping of the second terminal end of the elastic bandage strip after wrapping around the body part.

The absorbent pad can include a coagulant. The compression dressing can be rolled into a roll and packaged in a sterile packaging. The self-adhering elastic bandage strip can include an elastomeric substrate layer and an adhesive layer adhered thereto on one surface thereof. The absorbent pad can be affixed to a non-adhesive surface of the elastic bandage strip at the terminal end thereof.

The self-adhering elastic bandage strip can be selected having self-adherence properties so as to allow unrolling of a roll of the self-adhering elastic bandage strip without elastic extension of the elastic bandage strip. The self-adhering elastic bandage strip can be constructed so as to have self-adhesive properties sufficient to cause two adjacent layers of the compression dressing to remain adhered to one another under elastic extension without the use of a fastening mechanism.

The self-adhering elastic bandage strip can be constructed of a material including partially extended, longitudinally, vertically or irregularly extended elastic fibers bound to a fiber base to provide firm elastic extendibility in the longitudinal and vertical directions. The elastic bandage strip can be constructed of non-woven elastomeric fibers having firm elastic extendibility in longitudinal and vertical directions, and an adhesive, and can have self-adhesive properties sufficient to cause two adjacent layers of the bandage to remain adhered to one another without the use of a fastening mechanism. The self-adhering elastic bandage strip can be constructed of clear polyolefin polymer or copolymer films having firm elastic extendibility in the longitudinal and vertical directions, and having self-adhesive properties sufficient to cause two adjacent layers of the self-adhering elastic bandage strip to remain adhered to one another without the use of a fastening mechanism. The films can be printed polyolefin polymer or copolymer films. The self-adhesive elastic bandage strip can be constructed of a clear polyolefin polymer or copolymer film coated on one surface with an extendable, self-adhesive adhesive effective to cause two adjacent layers of the self-adhering elastic bandage strip to remain adhered to one another without the use of a fastening mechanism.

The absorbent pad can be affixed to the self-adhering elastic bandage strip by an affixing method selected from the group consisting of stitching, welding, needletacking and bonding. The bonding can be effected by an adhesive selected from a group consisting of natural rubber based adhesives and acrylic adhesives.

The absorbent pad can be constructed of foamed, woven or nonwoven material of natural or synthetic fibers selected from the group consisting of rayon, polyester, polyurethane, polyolefin, cellulose, cellulose derivatives, cotton, orlon and hydrogel polymeric materials.

The self-adhering elastic bandage strip can be stretched and secured with the blood migration markings indicating the migration of blood across the absorbent pad. The blood migration markings are visible through wrappings of the elastic bandage strip. The elastic bandage strip can create sufficient pressure to prevent exsanguination from an arterial wound without exceeding the pressure that would cause arterial blood flow to cease to a distal limb.

A method for the application of a compression dressing to a wound on a body part, includes the step of providing an elastic bandage strip that is elongated, transparent and self-adhering, and has first outer side and second inner side. The elastic bandage strip has a first unextended state and a second elastic extension state. A self-adhesive surface is provided on the elastic bandage strip. The self-adhesive surface can be a property of the material making up the elastic bandage strip or an adhesive coating applied to either the first outer side or second inner side that interacts with the opposing adjacent side after wrapping. The self-adhesive surface is capable of adhering the first side of the elastic bandage strip to the second side of an adjacent portion of the elastic bandage strip. An absorbent pad of lesser length than the elastic bandage strip is provided and is affixed to a terminal end of the elastic bandage strip. The elastic bandage strip is and of sufficient length such that the absorbent pad may be applied to the wound on the body part, and the remainder of the elastic bandage strip may be wrapped around the absorbent pad and the body part to hold the compression dressing in place and to exert a compressive force on the wound for a period of time to permit a therapeutic effect to the wound. At least one of the elastic bandage strip and the absorbent pad includes spaced apart markings for indicating the migration of blood across the absorbent pad. The blood migration and the indicia are visible through wrappings of the elastic bandage strip.

The absorbent pad is applied to the wound and the elastic bandage strip is wrapped around the body part to form adjacent wrapping layers. The self-adhesive on the first side of the elastic bandage strip adheres to the second side of adjacent wrapping layers, and the blood migration markings proving a reference visible through the wrapping layers of the migration of blood in the absorbent pad.

The self-adhering elastic bandage strip can be stretched and secured with the blood migration markings indicating the migration of blood across the absorbent pad. The blood migration markings are visible through wrappings of the elastic bandage strip. The elastic bandage strip creates sufficient pressure to prevent exsanguination from an arterial wound without exceeding the pressure that would cause arterial blood flow to cease to a distal limb.

A compression dressing includes an elastic bandage strip that is elongated, transparent and self-adhering, and has first outer side and second inner side. The elastic bandage strip has a first unextended state and a second elastically extended state. A self-adhesive surface on the elastic bandage strip is capable of adhering the first side of the elastic bandage strip to the second side of an adjacent portion of the elastic bandage strip.

An absorbent pad of lesser length than the elastic bandage strip is affixed to a terminal end of the elastic bandage strip. The elastic bandage strip is of sufficient length such that the absorbent pad may be applied to a wound on a body part, and the remainder of the elastic bandage strip may be wrapped around the absorbent pad and the body part to hold the compression dressing in place and to exert a compressive force on the wound for a period of time to permit a therapeutic effect to the wound. Compression markings are provided along a length of the elastic bandage strip. The compression markings have a first state when the elastic bandage strip is in the first unextended state, and the compression markings having a second state when the elastic bandage is in the second elastically extended state. The second state of the compression markings provides a visually perceptible indication of compression. The elastic bandage strip comprises a transparent material.

A method for the application of a compression dressing to a wound on a body part includesthe steps of providing an elastic bandage strip that is elongated and self-adhering, and has first outer side and second inner side, the elastic bandage strip having a first unextended state and a second elastically extended state; a self-adhesive surface on the elastic bandage strip, the self-adhesive surface capable of adhering the first side of the elastic bandage strip to the second side of an adjacent portion of the elastic bandage strip; an absorbent pad of lesser length than the elastic bandage strip, the absorbent pad being affixed to a terminal end of the elastic bandage strip, the elastic bandage strip being of sufficient length such that the absorbent pad may be applied to the wound on the body part, and the remainder of the elastic bandage strip may be wrapped around the absorbent pad and the body part to hold the compression dressing in place and to exert a compressive force on the wound for a period of time to permit a therapeutic effect to the wound.

Compression markings are provided along a length of the elastic bandage strip. The compression markings have a first state when the elastic bandage strip is in the first unextended state, and the compression markings having a second state when the elastic bandage is in the second elastically extended state. The second state of the compression markings provides a visually perceptible indication of compression.

The absorbent pad is applied to the wound and the elastic bandage strip is wrapped around the body part to form adjacent wrapping layers. The self-adhesive surface of the elastic bandage strip adheres the first side to the second side of adjacent wrapping layers, and the compression markings providing a reference for the elastic extension of and compression provided by the elastic bandage strip. The elastic bandage strip can include a transparent material. The elastic bandage strip can be stretched and secured with the compression markings indicating the pressure on the wound. The blood migration markings are visible through wrappings of the elastic bandage strip. The elastic bandage strip creates sufficient pressure to prevent exsanguination from an arterial wound without exceeding the pressure that would cause arterial blood flow to cease to a distal limb.

A compression dressing includes an elastic bandage strip that is elongated and self-adhering, and has first outer side and second inner side, the elastic bandage strip having a first unextended state and a second elastically extended state. A self-adhesive surface is provided on the the elastic bandage strip. The self-adhesive surface is capable of adhering the first outer side of the elastic bandage strip to the second inner side of an adjacent portion of the elastic bandage strip. An absorbent pad of lesser length than the elastic bandage strip is affixed at a terminal end of the elastic bandage strip and has an absorbent surface on the second side of the elastic bandage strip. The elastic bandage strip is of sufficient length such that the absorbent pad may be applied to a wound on a body part, and the remainder of the elastic bandage strip may be wrapped around the absorbent pad and the body part to hold the compression dressing in place and to exert a compressive force on the wound for a period of time to permit a therapeutic effect to the wound. A skin adhesive on the second inner side of and at the terminal end of the elastic bandage strip can be provided for adhering the terminal end of the elastic bandage strip to the skin of the patient. The elastic bandage strip can include a transparent material. A cover for the skin adhesive can also be provided.

A method for the application of a compression dressing to a wound on a body part includes the step of providing an elastic bandage strip that is elongated and self-adhering, and has first outer side and second inner side, the elastic bandage strip having a first unextended state and a second elastically extended state. A self-adhesive surface on the elastic bandage strip is capable of adhering the first side of the elastic bandage strip to the second side of an adjacent portion of the elastic bandage strip. An absorbent pad of lesser length than the elastic bandage strip is affixed to a terminal end of the elastic bandage strip. The elastic bandage strip is of sufficient length such that the absorbent pad may be applied to the wound on the body part, and the remainder of the elastic bandage strip may be wrapped around the absorbent pad and the body part to hold the compression dressing in place and to exert a compressive force on the wound for a period of time to permit a therapeutic effect to the wound. At least one of the elastic bandage strip and the absorbent pad include spaced apart markings for indicating the migration of blood across the absorbent pad, the blood migration and the indicia being visible through wrappings of the elastic bandage strip.

The absorbent pad is applied to the wound and wrapping the elastic bandage strip around the body part to form adjacent wrapping layers. The self-adhesive surface on the elastic bandage strip adheres the first side to the second side of adjacent wrapping layers, and the skin adhesive adheres the terminal end to the skin of the body part adjacent the wound to secure the terminal end to the body part for the subsequent wrapping of the wrapping layers. The elastic bandage strip can include a transparent material.

The self-adhering elastic bandage strip can be stretched and secured with the blood migration markings indicating the migration of blood across the absorbent pad. The blood migration markings are visible through wrappings of the elastic bandage strip. The elastic bandage strip creates sufficient pressure to prevent exsanguination from an arterial wound without exceeding the pressure that would cause arterial blood flow to cease to a distal limb.

### BRIEF DESCRIPTION OF THE DRAWINGS

There are shown in the drawings embodiments that are presently preferred it being understood that the invention is not limited to the arrangements and instrumentalities shown, wherein:

FIG. 1 is an exploded perspective view of a wound dressing according to the invention.

FIG. 2 is a perspective view, partially exploded and partially broken to indicate indefinite length.

FIG. 3 is a perspective view in a first mode of operation

FIG. 4 is a plan view of a first embodiment of a terminal end of the wound dressing.

FIG. 5 is a plan view of a second embodiment of a terminal end of the wound dressing.

FIG. 6 is a plan view of a third embodiment of a terminal end of the wound dressing.

FIG. 7 is a plan view of the absorbent pad of a terminal end of the wound dressing in a first stage of operation.

FIG. 8 is a plan view of the absorbent pad of a terminal end of the wound dressing in a second stage of operation.

FIG. 9 is a plan view of the absorbent pad of a terminal end of the wound dressing in a third stage of operation.

FIG. 10 is a plan view of compression markings in a first stage of operation.

FIG. 11 is a plan view of compression markings in a second stage of operation.

FIG. 12 is a plan view of compression markings in a third stage of operation.

FIG. 13 is a perspective view of a first step in applying a wound dressing according to the invention.

FIG. 14 is a perspective view of a second step in applying a wound dressing according to the invention.

FIG. 15 is a perspective view of a third step in applying a wound dressing according to the invention.

FIG. 16 is a perspective view of a fourth step in applying a wound dressing according to the invention.

FIG. 17 is a perspective view of a fifth step in applying a wound dressing according to the invention.

FIG. 18 is a perspective view of a sixth step in applying a wound dressing according to the invention.

FIG. 19 is a perspective view illustrating a terminal end securing device, in a first mode of operation.

FIG. 20 is a perspective view illustrating the terminal end securing device of FIG. 19, in a second mode of operation.

FIG. 21 is a perspective view illustrating an alternative terminal end securing device, in a first mode of operation.

FIG. 22 is a perspective view illustrating the alternative terminal end securing device of FIG. 21, in a second mode of operation.

### DETAILED DESCRIPTION OF THE INVENTION

A compression dressing includes an elastic bandage strip that is elongated, transparent and self-adhering, and has a first outer side and a second inner side. The elastic bandage strip has a first unextended state and a second elastically extended state. A self-adhesive surface is provided on the elastic bandage strip. The self-adhesive surface can be an adhesive applied to the elastic bandage strip or can be a property of the material making up the elastic bandage strip. The self-adhesive surface is capable of adhering the first side of the elastic bandage strip to the second side of an adjacent portion of the elastic bandage strip when wrapped. An absorbent pad of lesser length than the elastic bandage strip is affixed at or near a terminal end of the elastic bandage strip. The term ‘at or near the terminal end’ as used herein can mean that the absorbent pad is positioned at the terminal end or at a distance from the terminal end of within 25% of the total length of the elastic bandage strip. The absorbent pad can be positioned at a distance from the terminal end of the elastic bandage strip of within 1, 2, 3, 4, 5, 6, 7, 8, 9, 10, 11, 12, 13, 14, 15, 16, 17, 18, 19, 20, 21, 22, 23, 24 or 25% of the total length of the elastic bandage strip, or within a range of any high value or low value selected from these values.

The elastic bandage strip is designed, and of sufficient length such that the absorbent pad may be applied to a wound on a body part, and the remainder of the elastic bandage strip may be wrapped around the absorbent pad and the body part to hold the compression dressing in place and to exert a compressive force on the wound for a period of time to permit a therapeutic effect to the wound. At least one of the elastic bandage strip and the absorbent pad includes spaced apart blood migration markings for indicating the migration of blood across the absorbent pad. Alternatively, the blood migration markings can be provided on a separate clear layer that is adhered in place between the absorbent pad and the elastic bandage strip. The blood migration markings are visible through wrappings of the elastic bandage strip. The transparent material of the elastic bandage strip can comprise all or a portion of the elastic bandage strip.

The blood migration markings can vary, and many different types of such markings are possible. In one embodiment, the blood migration markings comprise a grid of columns and rows. Blood migration markings that are formed as concentric circles are possible. Other types of blood migration marking are possible. The compression dressing can further comprise indicia for identifying individual ones of the columns and rows. This will assist the user in appreciating and/or recording the migration of the blood over time. Many different kinds of such indicia are possible, including numerals and letters of the alphabet.

A method for the application and use of the compression dressing and the blood migration markings includes the step of applying the absorbent pad to the wound and wrapping the elastic bandage strip around the body part to form adjacent wrapping layers. The self-adhesive on the first side of the elastic bandage strip adheres to the second side of adjacent wrapping layers, and the blood migration markings proving a reference visible through the transparent wrapping layers of the migration of blood in the absorbent pad. This combination of transparent layers and blood migration markings allows simultaneous therapy and observation of the bleeding status.

Another embodiment of the compression dressing can include an elastic bandage strip that is elongated and self-adhering, and has a first outer side and a second inner side. The elastic bandage strip has a first unextended state and a second elastically extended state. A self-adhesive on the first side of the elastic bandage strip is capable of adhering the first side of the elastic bandage strip to the second side of an adjacent portion of the elastic bandage strip. An absorbent pad of lesser length than the elastic bandage strip is affixed at or near a terminal end of the elastic bandage strip. The elastic bandage strip is designed, and of sufficient length such that the absorbent pad may be applied to a wound on a body part, and the remainder of the elastic bandage strip may be wrapped around the adsorbent pad and the body part to hold the compression dressing in place and to exert a compressive force on the wound for a period of time to permit a therapeutic effect to the wound. Compression markings are provided along a length of the elastic bandage strip. The compression markings have a first state when the elastic bandage strip is in the first unextended state, and the compression markings have a second state when the elastic bandage is in the second elastic extension state. The second state of the compression markings provide a visually perceptible indication of stretch and compression. The elastic bandage strip can comprise a transparent material which will render the compression markings visible through adjacent wrapping layers.

The elastic and cohesive properties of the bandage allow for the creation of sufficient pressure to prevent exsanguination from an arterial wound without exceeding the pressure that would cause arterial blood flow to cease to the distal limb resulting in ischemia and damage, which would increase the chances that an amputation would be required. The desired stretching of the wrapping applied allows the effective pressure to be commensurate with the type of wound and severity of bleeding. The blood migration markings and/or compression markings, and transparent material making up the elastic bandage strip which allows the blood migration markings and compression markings to be readily visible, work together to achieve this optimal pressure which is critical to save both life and limb, as well as significantly extend the time such a device can remain on the patient during prolonged evacuation periods.

A method for the application of a compression dressing wherein at least one of the elastic bandage strip having the compression markings includes the step of applying the absorbent pad to the wound and wrapping the elastic bandage strip around the body part to form adjacent wrapping layers. The self-adhesive on the first side of the elastic bandage strip adheres to the second side of adjacent wrapping layers. The compression markings provide a reference for the elastic stretch of and compression provided by the elastic bandage strip.

Another embodiment of a compression dressing includes an elastic bandage strip that is elongated and self-adhering, and has a first outer side and a second inner side. The elastic bandage strip has a first unextended state and a second elastically extended state. A self-adhesive on the first outer side of the elastic bandage strip is capable of adhering the first outer side of the elastic bandage strip to the second inner side of an adjacent portion of the elastic bandage strip. An absorbent pad of lesser length than the elastic bandage strip is affixed at or near a terminal end of the elastic bandage strip and has an absorbent surface on the second side of the elastic bandage strip. The elastic bandage strip is designed, and of sufficient length such that the absorbent pad may be applied to a wound on a body part, and the remainder of the elastic bandage strip may be wrapped around the adsorbent pad and the body part to hold the compression dressing in place and to exert a compressive force on the wound for a period of time to permit a therapeutic effect to the wound. A skin adhesive on the second inner side of and at the terminal end of the elastic bandage strip is provided for adhering the terminal end of the elastic bandage strip to the skin of the patient. The elastic bandage strip can comprise a transparent material to permit viewing the absorbent pad and adjacent wrapping layers.

A method for the application of the compression dressing skin adhesive on the second inner side of and at the terminal end of the elastic bandage strip is provided for adhering the terminal end of the elastic bandage strip to the skin of the patient includes the step of applying the absorbent pad to the wound and wrapping the elastic bandage strip around the body part to form adjacent wrapping layers. The self-adhesive on the first side of the elastic bandage strip adheres to the second side of adjacent wrapping layers, and the skin adhesive adheres the terminal end of the elastic bandage strip to the skin of the body part adjacent the wound to secure the terminal end to the body part for the subsequent wrapping of the wrapping layers.

The elastic bandage strip is designed for exerting a compressive force when wrapped around a body part, sufficient to hold the compression dressing in place for a period of time to provide a therapeutic effect to a wound. The absorbent pad is affixed to the inner side of a terminal portion or in the alternative to a terminal end of strip. A portion of the strip which is not covered by the absorbent pad is adapted to directly contact a side of strip opposite of the absorbent pad or a back side of strip when the dressing is wrapped around the wounded body part. This contact allows the dressing to self-adhere or adhere to itself, prevents sliding or shifting of the wrap after it is in place, and provides a sufficient continuous compressive force to maintain a therapeutic effect on a bleeding wound to stop the bleeding, without the use of a fastening mechanism. For most of the length of elastic bandage strip, the absorbent pad does not cover the strip in order to provide a large, continuous contact area of the wrapped portion of strip. The adhesive side of the self-adhering elastic bandage strip can be positioned on the outer surface of the dressing, and a non-adhesive covering can be provided for the exposed surface of the absorbent pad.

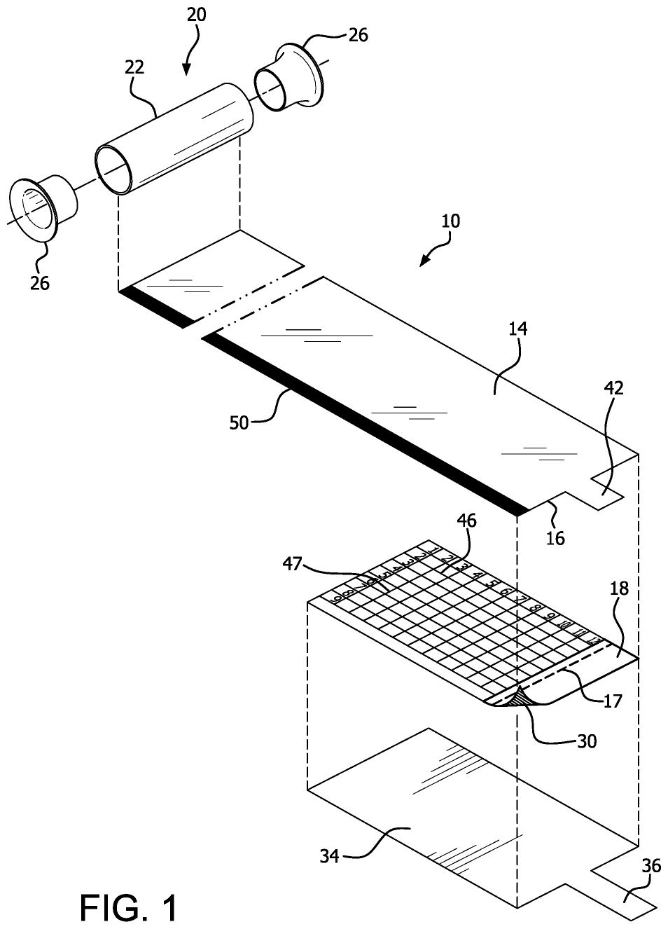

There is shown in FIGS. 1-18 a wound dressing 10 including an elastic bandage strip 14 having a terminal end 16 and an absorbent pad 18 that is secured to one side of the elastic bandage strip 14. The wound dressing 10 can also include a packaging 20 for the elastic bandage strip 14 and absorbent pad 18. The packaging 20 can take many forms, but in the embodiment shown has a winding tube 22 around which the elastic bandage strip 14 can be wound. The packaging 20 further can include end pieces 26 which can be rotatably secured to the ends of the winding tube 22 so as to rotate relative to the winding tube 22, facilitate gripping the end pieces 26 and unwinding the elastic bandage strip 14 from the winding tube 22.

An underside of the absorbent pad 18 can include an adhesive 30 for engaging the skin of the patient adjacent the wound. A cover 34 can initially be secured to the adhesive 30 so as to cover the absorbent pad 18 to prevent premature sticking and to maintain sterility until use. A tab 36 can be provided on the cover 34 which can be held by the user to assist in the removal of the cover 34 from its position adjacent the absorbent pad 18 and to assist in rapid deployment of the compression dressing. A tab 42 can be provided at the terminal end 16 of elastic bandage strip 14 to facilitate unwinding of the elastic bandage strip 14 from the winding tube 22. The tab 36 and the tab 42 can be positioned so as to be juxtaposed, and the tab 36 can have greater length in the tab 42 to facilitate gripping the tab 36 and removal of the cover 34.

The wound dressing 10 can include features to facilitate the proper application and use of the wound dressing 10. The absorbent pad 18 or an adjacent portion of the elastic bandage strip 14 that covers the wound can include blood migration markings such as rows 46 with indicia 48 and columns 47 with indicia 49 to delineate blood absorption into the absorbent pad 18 and progress or cessation of bleeding. Alternatively, the blood migration markings can be provided on a separate clear layer 17 that is adhered in place between the absorbent pad and the elastic bandage strip. Compression markings 50 can be used to provide an indication of the stretching of the elastic bandage strip 14 which can be related to the compressive force applied to the wound by the elastic bandage strip 14.

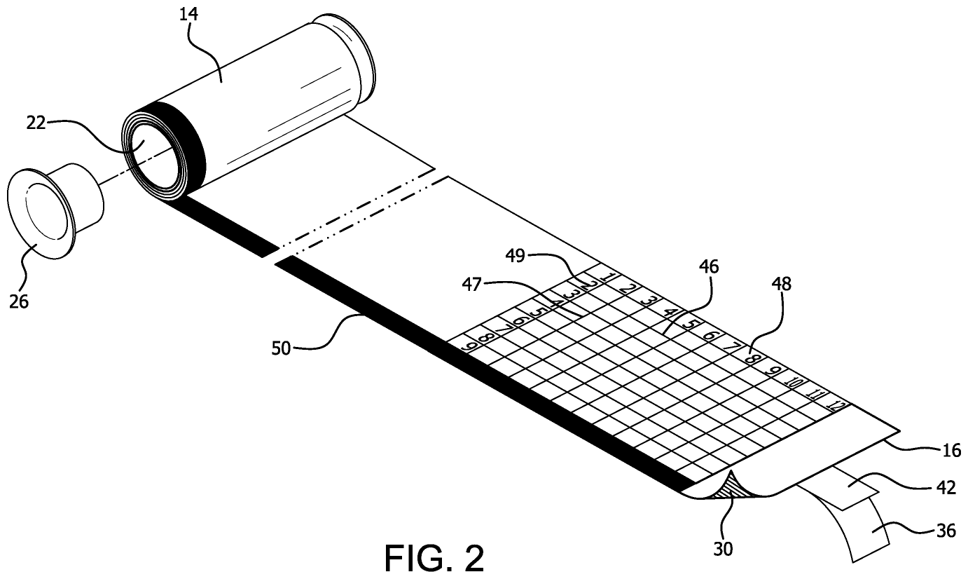

There is shown in FIG. 2 the elastic bandage strip 14 partially wound about the winding tube 22. In FIG. 3 the elastic bandage strip 14 and the absorbent pad 14 have been completely wound around the winding tube 22. The wound dressing 10 can be placed within a sterile packaging such as a flexible or rigid plastic container as are known and used in the art.

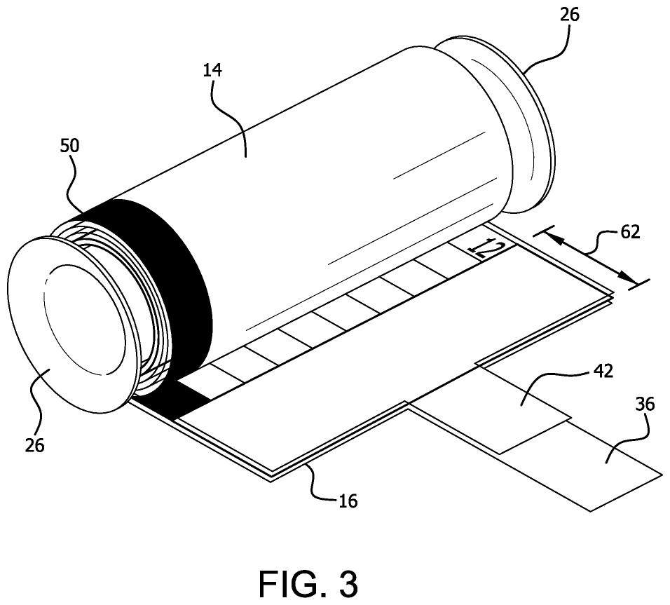

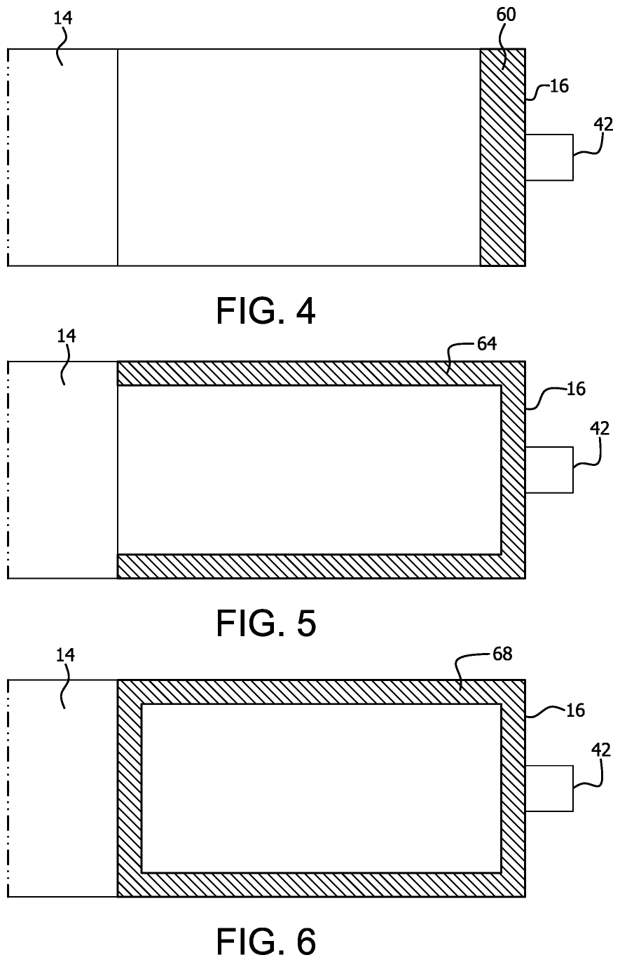

The adhesive 30 that is used to secure the elastic bandage strip 14 to the wound area can take different forms. In FIG. 4 the adhesive is an elongated strip 60 adjacent the terminal end 16 that has a width 62 (FIG. 3). In FIG. 5 the adhesive 64 is applied in a C-shaped pattern about the terminal edge 16 and sides of the elastic bandage strip 14. In FIG. 6 the adhesive 68 is applied in a parallelogram pattern. Other patterns are possible.

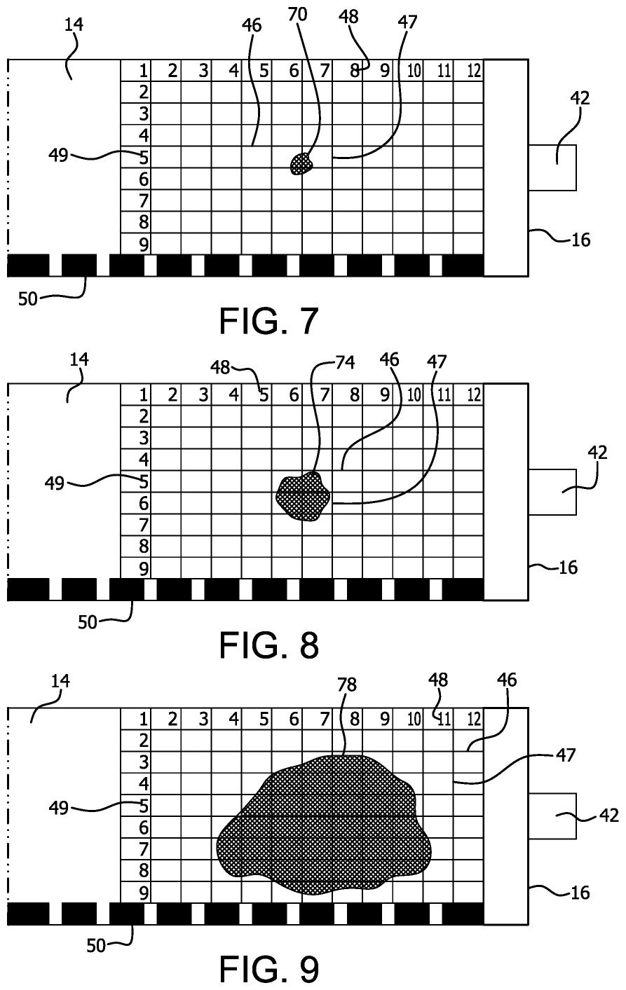

There is shown in FIGS. 7-9 the progress of blood migration from an initial stage in which absorbed blood is in an initial stage where the absorbed blood spot 70 does not cross two of the rows 46 or columns 47. In FIG. 8 there shown a subsequent stage in which the blood spot 74 is expanded from the dimensions of the initial blood spot 70. In FIG. 9 the blood spot 78 has increased in dimension, which indicates to the user that blood flow from the wound has continued and perhaps additional compression, another dressing, or further treatment is necessary. Other blood migration markings and indicia are possible.

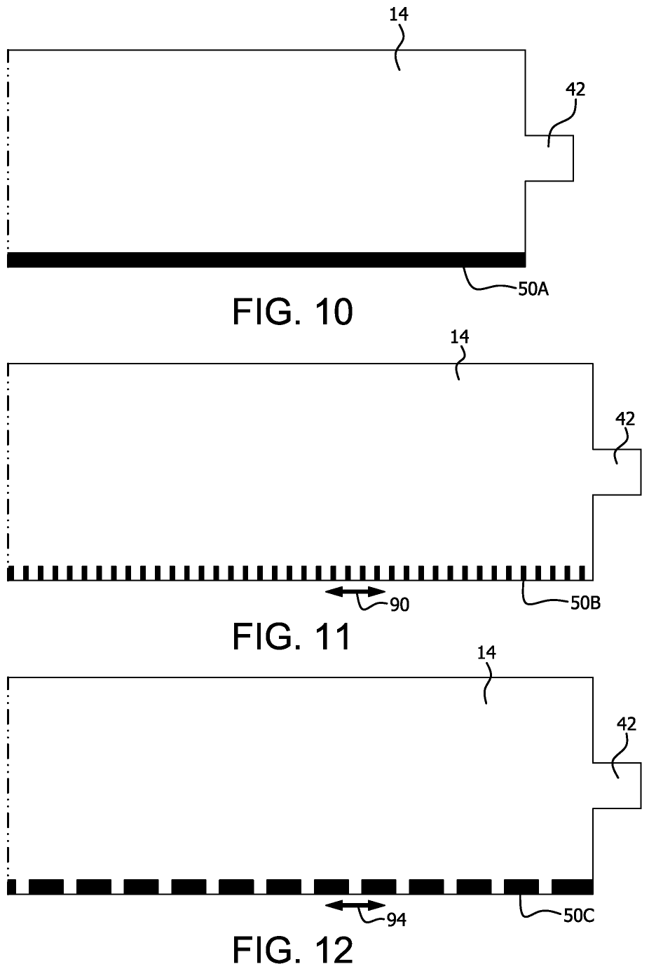

There shown in FIGS. 10-12 the use of compression markings 50 on the elastic bandage strip 14. In the initial un-stretched stage, the compression markings 50 A form essentially a solid line. In FIG. 11, with some stretching of the elastic bandage strip 14 shown by arrow 90, the individual compression markings 50 B are slightly separated. In FIG. 12, with further stretching indicated by arrow 94 the compression markings 50 C are further separated and stretched. The compression markings provide an indication of the stretching of the elastic bandage strip 14 which in turn relates to the amount of compression being applied to the wound because a component of the force has an inward direction toward the wound, and a component of the force is a traction force across the wound and these forces contribute to the tight wrapping of the compression dressing around the body part. This provides a relative indication to the user as an appropriate amount of compression for the wound. Other types of compression markings are possible.

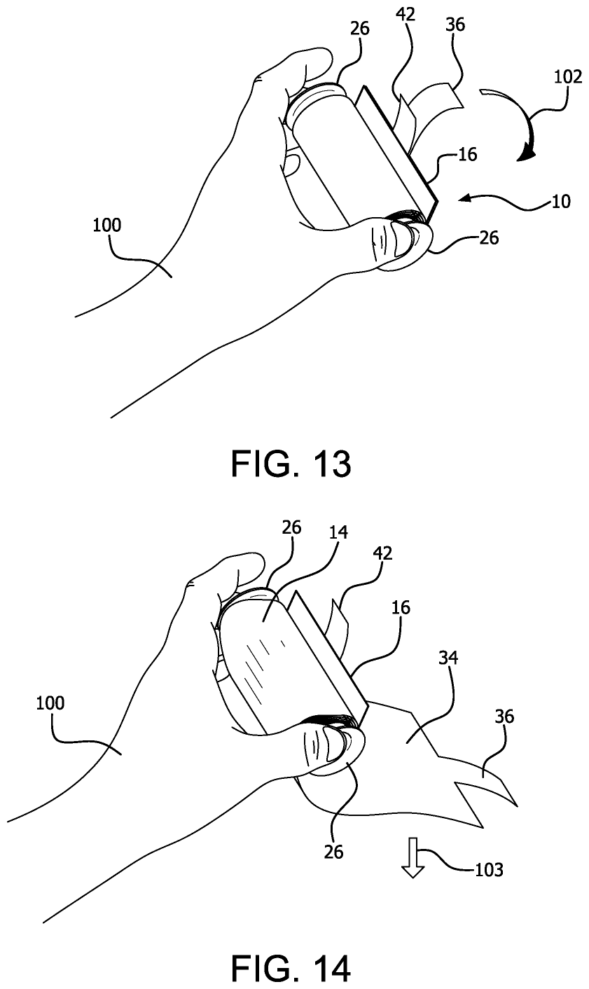

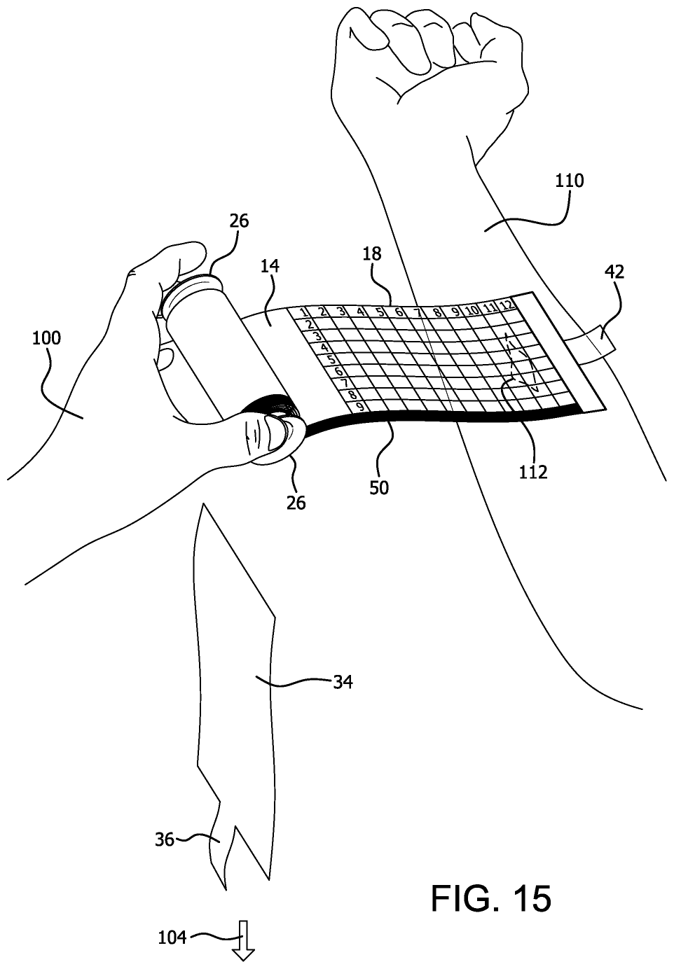

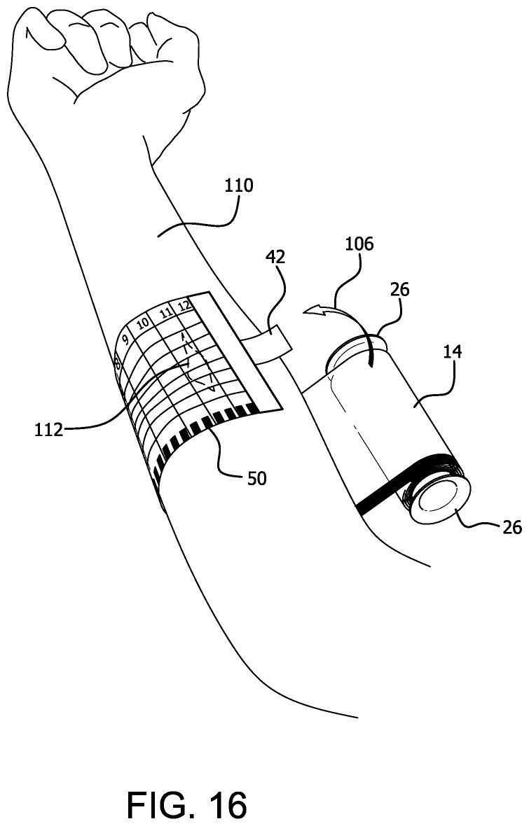

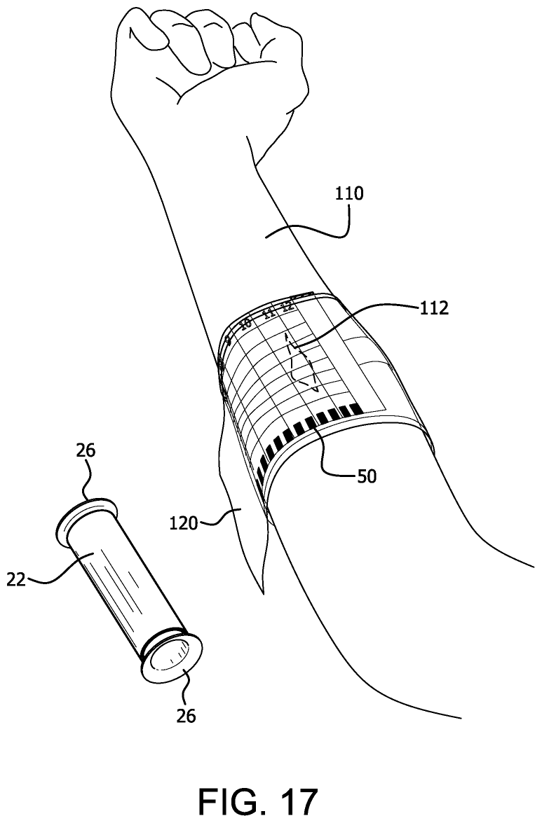

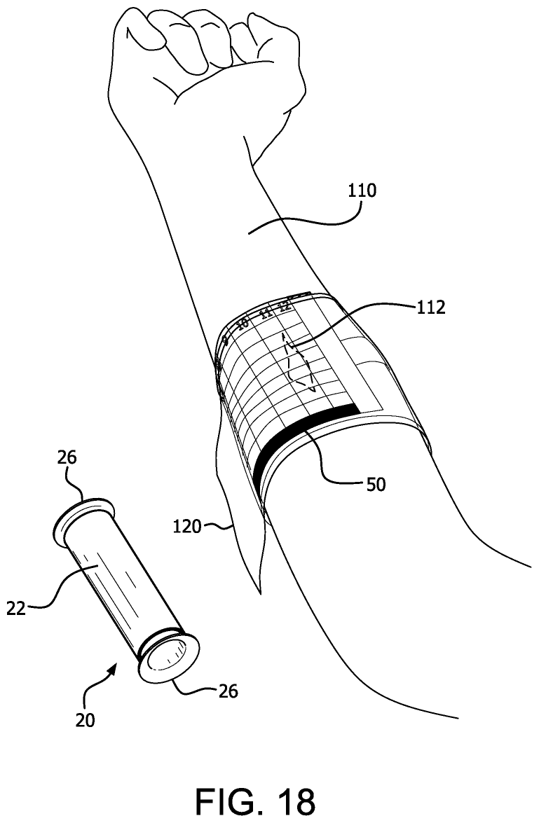

There shown in FIGS. 13-18 the application of the wound dressing 10 to a wound. The wound dressing 10 can be held in one hand 100 of the user with the thumb and one of the opposing fingers placed on the ends 26 (FIG. 13). The tab 36 is grasped with the other hand and pulled away from the tab 42 as shown by arrow 102. This will remove the cover 34 as shown by arrow 103 in FIG. 14. The cover 34 can be allowed to drop as shown by arrow 104 (FIG. 15). The adhesive 60 is used to cure the terminal end 16 to the forearm 110 with the absorbent pad 14 over the wound 112. The elastic bandage strip 14 is wrapped around the forearm 110 as shown by arrow 106 (FIG. 16). The wrapping is continued until the elastic bandage strip is completely wrapped around the forearm 110 or other subject body part and removed from the winding tube 22 (FIG. 17). The compression markings 50 are separated indicating that the elastic bandage strip 14 has been properly stretched to apply compression to the wound. The terminal end 120 distal to the absorbent pad 18 can be self-adhered to the portion of the elastic bandage strip 14 beneath it, or secured by other means. In FIG. 18 there is shown the condition where the compression markings 50 form essentially a solid line, and appear to be separated by little or no distance. This provides an indication to the user that an insufficient amount of stretch, and thereby compression to the wound, has been applied which otherwise would lead to unsatisfactory compression results.

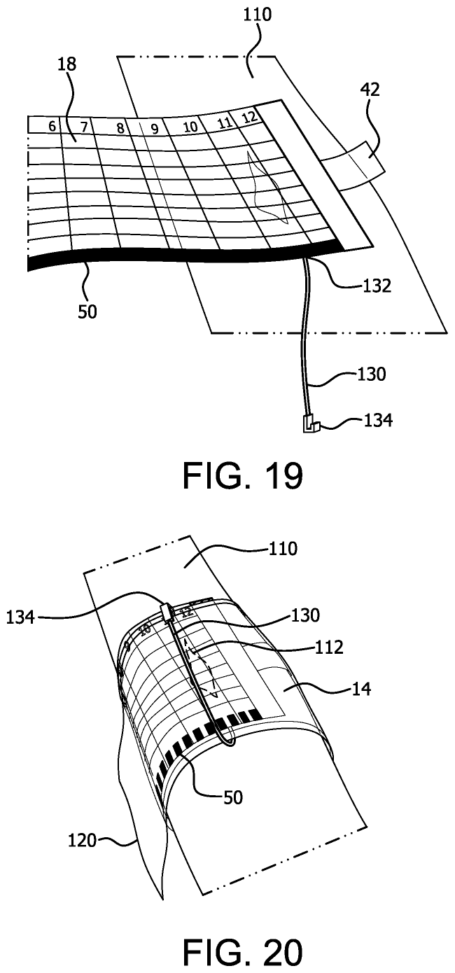

The terminal end 120 of the elastic bandage strip 14 distal to the terminal end 16 can self-adhere. It is also possible to provide structure to secure the terminal and 120 and other wrapping layers of the elastic bandage strip 14 once the wound dressing 10 has been properly positioned. One such structure shown in FIGS. 19-20 is a tethered clip comprising a tether 130 and clip 134. Initially, as shown in FIG. 19, the tethered clip has an end 132 secured to the elastic bandage strip 14 near the terminal end 16. The elastic bandage strip 14 is then wrapped as shown in FIG. 20. The tether 130 can be positioned over the wrapped layers and the clip 134 secured over the side edges of the wrapped layers to retain with the wrapped layers in position.

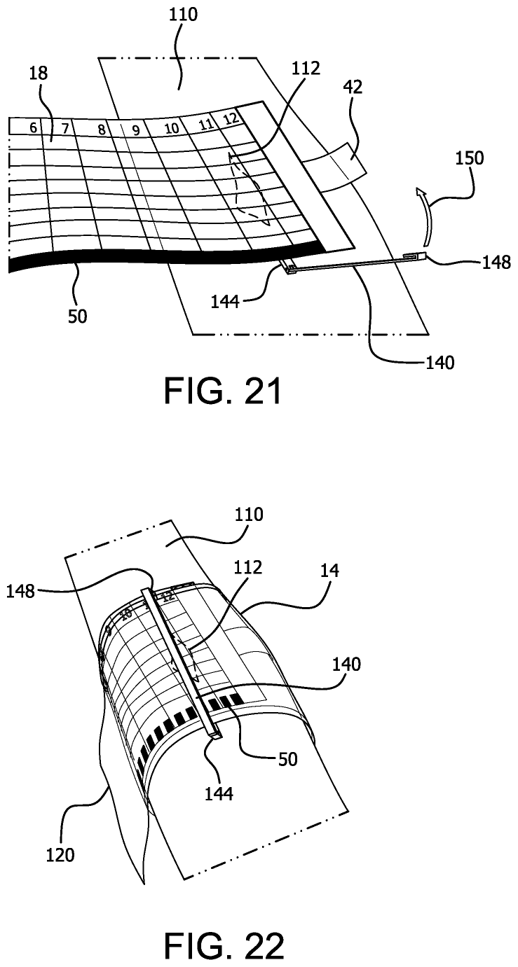

There is shown in FIGS. 21-22 an alternative structure for securing the wrapping layers of the elastic bandage in which a clip has a retaining arm 140 pivotably secured to a base 144 that is secured to the elastic bandage strip 14. A clip 148 is provided at a distal end of the retaining arm 140. In operation, after the elastic bandage strip 14 has been wrapped, the retaining arm 140 is pivoted in the direction shown by arrow 150 and the clip 148 is secured over side edges of the wrapped layers to retain the wrapped layers in position.

A preferred elastic bandage strip according to the present invention will provide a compressive force of between about 1 to 180 mm Hg in practice actual pressures averaging about 80 mm Hg are exerted when wrapped around a body part. This pressure is generally sufficient to provide a therapeutic benefit for a victim in the near shock state without necessarily restricting arterial blood flow to the point of ischemia distal to the bandaged body part. Therapeutic benefits provided by the present invention include control of bleeding, clot promotion, prevention of infection and prevention of further gaping of the wound margins. The dressing of the present invention is applicable onto body regions unprotected by personal security equipment such as bullet proof vests and helmets, and includes body locations where other compression dressings are inapplicable and/or ineffective. The wound dressing of the invention does not require fastening mechanisms other than self-adhesives, and the compression dressing of the present invention is therefore potentially readily self-applied by a single hand of an injured individual himself.

The self-adhering elastic bandage strip can be designed not to adhere to clothing, hair, skin or latex gloves. The self-adhering elastic bandage strip can be selected having self-adherence properties so as to allow unrolling of a roll of the elastic bandage strip without elastic extension of the elastic bandage strip. The self-adhering bandage strip can be constructed so as to have self-adhesive properties sufficient to cause two adjacent layers of the bandage to remain adhered to one another without the use of a fastening mechanism.

Materials suitable for use in elastic bandage strip of the present invention include materials which are elastic, conformable, provide adequate compression and which are self-adhering or which are made self-adhering by the addition of an adhesion surface thereon. Materials which may be sterilized, including radiation sterilized, are preferred.

The self-adhering bandage strip can be constructed of partially extended, longitudinally, vertically or irregularly extended elastic fibers bound to a fiber base to provide firm elastic extendibility in the longitudinal and vertical directions.

The self-adhering elastic bandage strip can be constructed of non-woven elastomeric fibers in embodiments where transparency is not necessary. Commercially available examples of elastic materials suitable for use in the present invention include woven bandages such as CUTTER-WRAP self-adhesive bandage (Cutter Animal Health, Miles Laboratories, Inc., Shawnee, Kans.), MEDI-RIP self-adherent bandage (Conco Medical Company, Bridgeport, Conn.) or SELF-GRIP sports tape/bandage (LMA, Ltd., South Norwalk, Conn.) and nonwoven bandages such as ROFLEX cohesive flexible bandage (Smith and Nephew Rolyan Inc., Menomonee Falls, Wis.), VET-FLEX veterinary flexible bandage (The Butler Company, Columbus, Ohio), CO-FLEX cohesive flexible bandage (Andover Coated Products, Inc., Marblehead, Mass.), FLEXUS support wrap (Kimberly-Clark Corporation Animal Care Division, Roswell, Ga.), COBAN self-adherent wrap (3M, St. Paul, Minn.), or EQUISPORT equine support. Preferred materials for use in the elastic bandage strip of the present invention are COBAN self-adherent elastomeric bandage and EQUISPORT equine support bandage. COBAN is a self-adherent material which does not adhere to skin or hair and which does not traumatize skin during the bandage wear period. EQUISPORT equine support bandage is a self-adherent material related to COBAN elastomeric bandage except that it is able to provide a greater amount of compression when used. Methods suitable to make these self-adherent materials are reported in U.S. Pat. No. 3,575,782 to Hansen and U.S. Pat. No. 4,984,584 to Hansen et al., both of which are incorporated by reference as if fully set forth herein. The increased compression is a result of using greater numbers of larger elastic strands in the material. For example, a material having elastic strands of about 550-1,700 denier and about 15-25 strands per inch is suitable.

A material made of melt blown microfiber webs may also be used in the strip of the present invention. The melt blown microfiber webs may be composed of a variety of well-known thermoplastic elastomers including polyurethane, styrene-isoprene block copolymer, styrene-butadiene block copolymer, (KRATON polymer, Shell Oil Company, Belpre, Ohio) and blends of these elastomers with polyolefins such as polypropylene and polyethylene. In addition, the melt blown microfiber webs may include, but are not limited to, staple fibers, such as rayon, polyester, nylon, cotton, LANSEAL fiber, cellulose, or polypropylene fibers, to provide a blend of elastomeric and staple fibers.

The self-adhering elastic bandage strip can be constructed of a transparent material. Thus, the dressing allows monitoring of continued bleeding even when applied, the blood migration markings, and/or the compression markings. Preferred materials are clear or printed polyolefin or copolymer films having firm extendibility in the longitudinal and vertical directions, and having self-adhesive properties. One such film that has been tested is a polyethylene film manufactured by Poleg Plastic Industries, Kibbutz Gevim, Israel, consisting of 54% low-density polyethylene (IPETHENE, Carmel Olefins Ltd., Haifa, Israel), 40% copolymer of ethylene and octene-1, and 6% SEBS-copolymer. This film has a thickness of 100 microns, modulus at 25% elongation of 0.6 (M.D.) and 0.5 (C.D.) Kg/mm², tensile strength of 2.0 (M.D.) and 2.8 (C.D.) Kg/mm², elongation at break of 600% (M.D.) and 1000% (C.D.), adhesion strength of 1600 gram/10 cm and adhesion to steel of 16.4 Newtons/inch. Another film which has been tested is AFFINITY* KC 8852 Polyolefin Plastomer (DOW Chemical, Horgen, Switzerland).

The clear polyolefin polymer or copolymer film can be coated with an extendable, self-adhesive finish. Thus, the self-adhering properties of polyolefin films may be enhanced by coating these materials with natural rubber latex or latex free adhesives having low tack, providing greater adhesive properties while maintaining non-adherence to clothing, skin, hair or latex gloves. The latex or low tack adhesive is preferably coated onto one or both sides of the clear film in the amount of about 0.01-75 wt. % in order to provide for self-adherence or self-bonding when the strip is wrapped around a wound.

The absorbent pad can be attached to the self-adhering elastic bandage strip by an attaching method such as, but not limited to, stitching, welding, needletacking or bonding. Suitable adhesives which may be used to bind an absorbent pad to an elastic bandage strip of the present invention include pressure sensitive adhesives such as polyacrylates, polyvinylethers, and poly alpha-olefins, as well as polymers which may be formulated with appropriate tackifiers such as natural rubber, styrene-isoprene block copolymer, silicone rubber, cis-polyisoprene, styrene butadiene, and cis-polybutadiene, hot-melt adhesives such as low-density polyethylene, ethylene-vinyl acetate copolymers, ethylene-ethyl acrylate copolymers, paraffin waxes, polyesters, polyamides, polypropylene, styrene-butadiene block copolymers and polyurethanes, and curable adhesives such as silicones and urethanes. In addition, repositionable adhesives such as microsphere pressure sensitive adhesives may be used to bond the absorbent pad to the elastic bandage strip. Highly preferred adhesives would be those which are biocompatible with skin and which generally do not cause irritation or undesirable sensitivity reactions when in contact with skin for extended periods of time under normal conditions such as natural rubber and acrylate based adhesives.

The absorbent pad can be constructed of foamed, woven or non-woven material of natural or synthetic fibers, such as, but not limited to, rayon, polyester, polyurethane, polyolefin, cellulose, cellulose derivatives, cotton, orlon or hydrogel polymeric materials. Preferably, the absorbent material both absorbs wound exudate and protects the wound by absorbing shocks. Other types of materials having similar absorbent properties and characteristics would also be suitable for use in this invention. Examples of absorbent materials are dacron-polyester cast padding MW0.4, (3M, St. Paul, Minn.), DELTA-ROL acrylic cast padding 6884, (Johnson & Johnson, New Brunswick, N.J.), SOF-ROL 100% needle-tacked rayon cast padding HRI 8137-009034, (Johnson & Johnson, New Brunswick, N.J.), SPECIALIST cotton cast padding HRI 8137-009044 (Johnson & Johnson, New Brunswick, N.J.), WEBRIL cotton undercast padding 3175 (The Kendall Company, Boston, Mass.), WEBRIL II cotton undercast padding 4221 (The Kendall Company, Boston, Mass.), nonwoven cotton web 142-451 and nonwoven rayon/polyester web 140-037 (VeraTec Company, Wapole, Mass.), and an absorbent resilient open-cell foam such as polyurethane, polyester, polyether, polyolefin foams as described in U.S. Pat. No. 3,908,645 to Sandvig.

Other suitable absorbent materials include composite materials such as nonwoven polymeric matrices combined with highly hydrophilic fluid absorbing materials. Highly hydrophilic fluid absorbing materials include polymeric absorbent fibers or particles, such as, but not limited to, modified polysaccharides, modified polyurethanes and high molecular weight acrylic polymers containing hydrophilic groups. A preferred highly hydrophilic fluid absorbing material is acrylonitrile fibers treated with alkali metal hydroxides. A commercially available hydrogel polymeric material is distributed under the tradename LANSEAL fiber (Japan Exlan Co., Ltd., Osaka, Japan). These types of composite absorbent materials are readily prepared using well known methods such as the method reported in U.S. Pat. No. 4,957,795 to Riedel.

Typically, the absorbent material includes one or more layers of a nonwoven, melt blown absorbent fiber which provides loft to the material and which absorbs liquids. The surface of the absorbent material which contacts the wound may additionally be treated or modified so that it will not adhere to the wound. For example, the absorbent material may be covered with a variety of commercially available wound contact materials such as TEGAPORE woven nylon web, TEGADERM polyurethane film or TEGASORB hydrocolloid (all available from 3M, St. Paul, Minn.) as well as other well-known related materials. Preferably, the absorbent pad includes a coagulant, enhancing clot formation in the wound and improving bleeding control properties of the compression dressing.

As used herein in the specification and in the claims section that follows, the term “coagulant” is defined as any agent that promotes clotting, or coagulation of blood, which may be safely applied to an open wound. A non-limiting example of such a coagulant material comprising gelatin, thrombin and calcium is described in U.S. Pat. No. 6,045,570 to Epstein, et al.

The compression dressing can be rolled into a roll and packaged in a sterile packaging. The dressing may be sterilized by, for example, exposure to heat, radiation or gas.

According to another aspect of the present invention, there is provided a method of preparation and packaging of the compression dressing of the invention to enable rapid application within conditions of limited space, the method is effected by rolling the self-adhesive elastic bandage strip without affording contact between self-adherent surfaces, and affixing the absorbent pad to the inner side of the terminal portion or to the terminal end of the self-adhering bandage strip. It will be appreciated in this respect that, standard issue military (“first-aid”) wound bandages, constructed with the absorption pad in the center of fastening strings, for example, must be spread open and extended to achieve desired wound coverage and compression, making their use in confined space awkward and ineffective.

According to yet another aspect of the present invention there is provided a method of preparation and packaging of the compression dressing to enable easier application. This method is effected by rolling and packaging the self-adhering elastic bandage strip with its adhesive surface on an outer, exposed side of a rolled self-adhering elastic bandage strip. In this case, a non-adhesive covering is provided for the surface of the absorbent pad, and the adhesive properties of the self-adhering elastic bandage strip are selected so as not to adhere to, for example, latex gloves and/or packaging materials, the advantages being, easier application.

When prepared and packaged in the above-mentioned manner, the compression dressing of the invention may be unwound and wrapped while still in rolled form, within a confined space around a wounded body part, affording more rapid and effective treatment in emergency and trauma situations.

According to a further aspect of the present invention there is provided a method of application of the compression dressing of the invention over a wound in a body part. The method according to this aspect of the present invention is performed by positioning the absorbent pad over the wound so as to effectively cover the wound; orienting the self-adhering elastic bandage strip substantially in continuation or perpendicularly to a length of the absorbent pad covering the wound and wrapping the body part, covering at least the absorbent pad. Additionally, and alternatively, there is provided a method of rapid application of the compression dressing of the invention over a wound in a body part, the method effected by positioning absorbent pad over the wound so as to effectively cover the wound; orienting the self-adhering elastic bandage strip substantially in continuation or perpendicularly to a length of the absorbent pad covering the wound; rotating the rolled self-adhering elastic bandage strip 180 degrees around it's longitudinal axis, causing the self-adhesive surface to face outward; and wrapping the body part, covering at least the absorbent pad. When applied in this manner, the outward-facing orientation of the self-adhering surface allows for greater tension and more effective compression over the wounded body part.

The invention provides an efficient method for dressing “entrance-exit wounds”. A body part, such as a limb, may sustain wounds on opposite surfaces resulting from penetration injuries, for example, gunshot wounds. The addition of absorbent pad or pads to the wound opposite (e.g. the “exit” wound) the absorbent pad of the compression dressing (e.g. covering the “entrance” wound), and compression of both wounds with the same self-adhering elastic bandage strip allows for rapid and efficient dressing and bleeding control of these often difficult-to-manage trauma.

The compression dressing of the invention may be easily applied by untrained personnel or even, in some instances by the injured individual himself. The dressing is not intended to absorb large amounts of blood, but allows hygienic isolation of the area and prevents excess bleeding by sealing the wounded region and compressing and thereby blocking ruptured blood vessels. Superior compression of the wounded body part is achieved by reduction of space between the elastic strip and the wound, the degree of compression determined by altering the amount of extension and tightening of the bandage during wrapping as chosen at the discretion, and according to the experience, of the applicator. Thus, the compression dressing of the present invention affords a wide range of possible pressures, as may suit demands, from moderate adherence in treating, for example, flesh wounds or bums, to complete obstruction of local and distal circulation, as in deep wounds with severe arterial bleeding.

The invention provides a method of application of the compression dressing of the invention over a longitudinal wound in a body part, the longitudinal wound exceeding a width but not a length of the absorbent pad. The method according to this aspect of the present invention is performed by positioning the absorbent pad over the longitudinal wound so as to effectively cover the wound; orienting the elastic bandage strip substantially perpendicularly to a length of the absorbent pad covering the wound; and wrapping the body part, covering at least an entire length of the absorbent pad. A standard military bandage cannot be easily deployed along the length of a limb. Thus, the effective dressing of a large longitudinal wound in a limb, for example, requires opening and application of more than one bandage of this kind. By simply turning the elastic bandage strip of the present invention perpendicular to the absorbent pad before wrapping, longitudinal wounds up to the entire length of the absorbent pad may be quickly and effectively treated.

The invention as shown in the drawings and described in detail herein disclose arrangements of elements of particular construction and configuration for illustrating preferred embodiments of structure and method of operation of the present invention. It is to be understood however, that elements of different construction and configuration and other arrangements thereof, other than those illustrated and described may be employed in accordance with the spirit of the invention, and such changes, alternations and modifications as would occur to those skilled in the art are considered to be within the scope of this invention as broadly defined in the appended claims. In addition, it is to be understood that the phraseology and terminology employed herein are for the purpose of description and should not be regarded as limiting.

We claim:

1. A compression dressing, comprising:
an elastic bandage strip that is elongated, transparent and self-adhering, and has first outer side and second inner side, the elastic bandage strip having a first unextended state and a second elastically extended state;
a self-adhesive surface on the elastic bandage strip, the self-adhesive surface being capable of adhering the first outer side of the elastic bandage strip to the second inner side of an adjacent portion of the elastic bandage strip when wrapped;
an absorbent pad of lesser length than the elastic bandage strip, the absorbent pad being affixed to a terminal end of the elastic bandage strip, the elastic bandage strip being of sufficient length such that the absorbent pad may be applied to a wound on a body part, and the remainder of the elastic bandage strip may be wrapped around the adsorbent pad and the body part to hold the compression dressing in place and to exert a compressive force on the wound for a period of time to permit a therapeutic effect to the wound; and,
spaced apart blood migration markings for indicating the migration of blood across the absorbent pad, the blood migration markings being visible through wrappings of the elastic bandage strip.

2. The compression dressing of claim 1, wherein the blood migration markings are provided on at least one selected from the group consisting of the elastic bandage strip, the absorbent pad, and a blood migration marking layer between the absorbent pad and the elastic bandage strip.

3. The compression dressing of claim 2, wherein the blood migration markings comprise a grid of columns and rows.

4. The compression dressing of claim 3, further comprising indicia for identifying individual ones of the columns and rows.

5. The compression dressing of claim 1, wherein the elastic bandage strip comprises a first terminal end affixed to the absorbent pad, and a second terminal end, the compression dressing further comprising a fastening device secured nearer to the first terminal end than to the second terminal end, for engaging the second terminal end to the first terminal end and preventing unwrapping of the second terminal end of the elastic bandage strip after wrapping around the body part.

6. The compression dressing of claim 1, wherein the absorbent pad includes a coagulant.

7. The compression dressing of claim 1, rolled into a roll and packaged in a sterile packaging.

8. The compression dressing of claim 1, wherein the self-adhering elastic bandage strip includes an elastomeric substrate layer and an adhesive layer adhered thereto on one surface thereof, the absorbent pad being affixed to a non-adhesive surface of the elastic bandage strip at the terminal end thereof.

9. The compression dressing of claim 1, wherein the self-adhering elastic bandage strip is selected having self-adherence properties so as to allow unrolling of a roll of the self-adhering elastic bandage strip without elastic extension of the elastic bandage strip.

10. The compression dressing of claim 1, wherein the self-adhering elastic bandage strip is constructed so as to have self-adhesive properties sufficient to cause two adjacent layers of the compression dressing to remain adhered to one another under elastic extension without the use of a fastening mechanism.

11. The compression dressing of claim 1, wherein the self-adhering elastic bandage strip is constructed of a material including partially extended, longitudinally, vertically or irregularly extended elastic fibers bound to a fiber base to provide firm elastic extendibility in the longitudinal and vertical directions.

12. The compression of claim 1, wherein the elastic bandage strip is constructed of non-woven elastomeric fibers having firm elastic extendibility in longitudinal and vertical directions, and an adhesive, and having self-adhesive properties sufficient to cause two adjacent layers of the bandage to remain adhered to one another without the use of a fastening mechanism.

13. The compression dressing of claim 1, wherein the self-adhering elastic bandage strip is constructed of clear polyolefin polymer or copolymer films having firm elastic extendibility in the longitudinal and vertical directions, and having self-adhesive properties sufficient to cause two adjacent layers of the self-adhering elastic bandage strip to remain adhered to one another without the use of a fastening mechanism.

14. The compression dressing of claim 13, wherein the films are printed polyolefin polymer or copolymer films.

15. The compression dressing of claim 1, wherein the self-adhesive elastic bandage strip is constructed of a clear polyolefin polymer or copolymer film coated on one surface with an extendable, self-adhesive adhesive effective to cause two adjacent layers of the self-adhering elastic bandage strip to remain adhered to one another without the use of a fastening mechanism.

16. The compression dressing of claim 1, wherein the absorbent pad is affixed to the self-adhering elastic bandage strip by an affixing method selected from the group consisting of stitching, welding, needletacking and bonding.

17. The compression dressing of claim 16, wherein the bonding is effected by an adhesive selected from a group consisting of natural rubber based adhesives and acrylic adhesives.

18. The compression dressing of claim 1, wherein the absorbent pad is constructed of foamed, woven or nonwoven material of natural or synthetic fibers selected from the group consisting of rayon, polyester, polyurethane, polyolefin, cellulose, cellulose derivatives, cotton, orlon and hydrogel polymeric materials.

19. The compression dressing of claim 1, wherein the self-adhering elastic bandage strip is stretched and secured with the blood migration markings indicating the migration of blood across the absorbent pad, the blood migration markings being visible through wrappings of the elastic bandage strip, to create sufficient pressure to prevent exsanguination from an arterial wound without exceeding the pressure that would cause arterial blood flow to cease to a distal limb.

20. A method for the application of a compression dressing to a wound on a body part, comprising the steps of:
providing an elastic bandage strip that is elongated, transparent and self-adhering, and has first outer side and second inner side, the elastic bandage strip having a first unextended state and a second elastic extension state; a self-adhesive surface on the elastic bandage strip, the self-adhesive surface being capable of adhering the first side of the elastic bandage strip to the second side of an adjacent portion of the elastic bandage strip; an absorbent pad of lesser length than the elastic bandage strip, the absorbent pad being affixed to a terminal end of the elastic bandage strip, the elastic bandage strip being of sufficient length such that the absorbent pad may be applied to the wound on the body part, and the remainder of the elastic bandage strip may be wrapped around the absorbent pad and the body part to hold the compression dressing in place and to exert a compressive force on the wound for a period of time to permit a therapeutic effect to the wound; wherein at least one selected from the group consisting of the elastic bandage strip, the absorbent pad, and a layer between the elastic bandage strip and the absorbent pad, includes spaced apart blood migration markings for indicating the migration of blood across the absorbent pad, the blood migration markings and the indicia being visible through wrappings of the elastic bandage strip;
applying the absorbent pad to the wound and wrapping the elastic bandage strip around the body part to form adjacent wrapping layers, the self-adhesive surface on the elastic bandage strip adhering the first side to the second side of adjacent wrapping layers, and the blood migration markings proving a reference visible through the wrapping layers of the migration of blood in the absorbent pad.

21. The method of claim 20, wherein the self-adhering elastic bandage strip can be stretched and secured with the blood migration markings indicating the migration of blood across the absorbent pad, the blood migration markings being visible through wrappings of the elastic bandage strip, to create sufficient pressure to prevent exsanguination from an arterial wound without exceeding the pressure that would cause arterial blood flow to cease to a distal limb.

22. A compression dressing, comprising,
an elastic bandage strip that is elongated, transparent and self-adhering, and has first outer side and second inner side, the elastic bandage strip having a first unextended state and a second elastically extended state;
a self-adhesive on the elastic bandage strip, the self-adhesive surface being capable of adhering the first side of the elastic bandage strip to the second side of an adjacent portion of the elastic bandage strip;
an absorbent pad of lesser length than the elastic bandage strip, the absorbent pad being affixed to a terminal end of the elastic bandage strip, the elastic bandage strip being of sufficient length such that the absorbent pad may be applied to a wound on a body part, and the remainder of the elastic bandage strip may be wrapped around the absorbent pad and the body part to hold the compression dressing in place and to exert a compressive force on the wound for a period of time to permit a therapeutic effect to the wound; and,
wherein compression markings are provided along a length of the elastic bandage strip, the compression markings having a first state when the elastic bandage strip is in the first unextended state, and the compression markings having a second state when the elastic bandage is in the second elastically extended state, the second state of the compression markings providing a visually perceptible indication of compression.

23. The compression dressing of claim 22, wherein the elastic bandage strip comprises a transparent material.

24. A method for the application of a compression dressing to a wound on a body part, comprising the steps of:
providing an elastic bandage strip that is elongated and self-adhering, and has first outer side and second inner side, the elastic bandage strip having a first unextended state and a second elastically extended state; a self-adhesive surface on the elastic bandage strip, the self-adhesive surface being capable of adhering the first side of the elastic bandage strip to the second side of an adjacent portion of the elastic bandage strip; an absorbent pad of lesser length than the elastic bandage strip, the absorbent pad being affixed to a terminal end of the elastic bandage strip, the elastic bandage strip being of sufficient length such that the absorbent pad may be applied to the wound on the body part, and the remainder of the elastic bandage strip may be wrapped around the absorbent pad and the body part to hold the compression dressing in place and to exert a compressive force on the wound for a period of time to permit a therapeutic effect to the wound;
wherein compression markings are provided along a length of the elastic bandage strip, the compression markings having a first state when the elastic bandage strip is in the first unextended state, and the compression markings having a second state when the elastic bandage is in the second elastically extended state, the second state of the compression markings providing a visually perceptible indication of compression.
applying the absorbent pad to the wound and wrapping the elastic bandage strip around the body part to form adjacent wrapping layers, the self-adhesive surface on the elastic bandage strip adhering the first side to the second side of adjacent wrapping layers, and the compression markings providing a reference for the elastic extension of and compression provided by the elastic bandage strip.

25. The method of claim 24, wherein the elastic bandage strip comprises a transparent material.

26. The method of claim 25, wherein the elastic bandage strip is stretched and secured with the compression markings indicating the pressure on the wound, the blood migration markings being visible through wrappings of the elastic bandage strip, to create sufficient pressure to prevent exsanguination from an arterial wound without exceeding the pressure that would cause arterial blood flow to cease to a distal limb.

27. A compression dressing, comprising,
an elastic bandage strip that is elongated and self-adhering, and has first outer side and second inner side, the elastic bandage strip having a first unextended state and a second elastically extended state;
a self-adhesive surface on the elastic bandage strip, the self-adhesive surface being capable of adhering the first outer side of the elastic bandage strip to the second inner side of an adjacent portion of the elastic bandage strip;
an absorbent pad of lesser length than the elastic bandage strip, the absorbent pad being affixed at a terminal end of the elastic bandage strip and having an absorbent surface on the second side of the elastic bandage strip, the elastic bandage strip being of sufficient length such that the absorbent pad may be applied to a wound on a body part, and the remainder of the elastic bandage strip may be wrapped around the absorbent pad and the body part to hold the compression dressing in place and to exert a compressive force on the wound for a period of time to permit a therapeutic effect to the wound; and,
a skin adhesive on the second inner side of and at the terminal end of the elastic bandage strip for adhering the terminal end of the elastic bandage strip to the skin of the patient.

28. The compression dressing of claim 27, wherein the elastic bandage strip comprises a transparent material.

29. The compression dressing of claim 27, further comprising a cover for the skin adhesive.

30. A method for the application of a compression dressing to a wound on a body part, comprising the steps of:
providing an elastic bandage strip that is elongated and self-adhering, and has first outer side and second inner side, the elastic bandage strip having a first unextended state and a second elastically extended state; a self-adhesive surface on the elastic bandage strip, the self-adhesive surface being capable of adhering the first side of the elastic bandage strip to the second side of an adjacent portion of the elastic bandage strip; an absorbent pad of lesser length than the elastic bandage strip, the absorbent pad being affixed to a terminal end of the elastic bandage strip, the elastic bandage strip being of sufficient length such that the absorbent pad may be applied to the wound on the body part, and the remainder of the elastic bandage strip may be wrapped around the absorbent pad and the body part to hold the compression dressing in place and to exert a compressive force on the wound for a period of time to permit a therapeutic effect to the wound; wherein at least one selected from the group consisting of the elastic bandage strip, the absorbent pad, and a layer between the elastic bandage strip and absorbent pad, includes spaced apart blood migration markings for indicating the migration of blood across the absorbent pad, the blood migration and the blood migration markings being visible through wrappings of the elastic bandage strip; and,
applying the absorbent pad to the wound and wrapping the elastic bandage strip around the body part to form adjacent wrapping layers, the self-adhesive surface on the elastic bandage strip adhering the first side to the second side of adjacent wrapping layers, and the skin adhesive adhering the terminal end to the skin of the body part adjacent the wound to secure the terminal end to the body part for the subsequent wrapping of the wrapping layers.

31. The method of claim 30, wherein the elastic bandage strip comprises a transparent material.

32. The method of claim 31, wherein the self-adhering elastic bandage strip can be stretched and secured with the blood migration markings indicating the migration of blood across the absorbent pad, the blood migration markings being visible through wrappings of the elastic bandage strip, to create sufficient pressure to prevent exsanguination from an arterial wound without exceeding the pressure that would cause arterial blood flow to cease to a distal limb.
